(19) United States
(12) Patent Application Publication
(10) Pub. No.: US 2025/0221417 A1
(43) Pub. Date: Jul. 10, 2025

(54) SYMBIOTIC FUNGAL STRAIN FOR PROMOTING GERMINATION OF CREMASTRA GENUS PLANT SEEDS AND APPLICATION THEREOF

(71) Applicant: WEST ANHUI UNIVERSITY, LU'AN (CN)

(72) Inventors: BANGXING HAN, LU'AN (CN); SHANYONG YI, LU'AN (CN); TAO XU, LU'AN (CN)

(73) Assignee: WEST ANHUI UNIVERSITY, LU'AN (CN)

(21) Appl. No.: 18/991,334

(22) Filed: Dec. 20, 2024

(30) Foreign Application Priority Data
Jan. 4, 2024 (CN) 2024100136166

(51) Int. Cl. A01N 63/30; A01P 21/00; C12N 1/14; C12Q 1/6895

(52) U.S. Cl. CPC A01N 63/30; A01P 21/00; C12N 1/145; C12Q 1/6895

(57) ABSTRACT

The present invention relates to the field of biotechnology and provides a symbiotic fungal strain for promoting germination of Cremastra genus plant seeds and application thereof. The strain is isolated and purified from soil symbiotic fungi of Cremastra appendiculata (D. Don) Makino. Through ITS sequence sequencing, it is identified as a fungus of the genus Coprinellus, with the taxonomic name Coprinellus sp. (Coprinellus genus), and its preservation number is CCTCC_NO:M2023983. Symbiotic germination experiments conducted by co-cultivating the strain with Cremastra genus plant seeds demonstrated that the strain can significantly enhance seed germination rates.

### CROSS REFERENCE TO RELATED DOCUMENTS

The present application includes a sequence listing named: “BSSWO002US-SQE LISTING XML.xml”, created on Mar. 25, 2025, in the size of 12,022 bytes, which is incorporated herein by reference in its entirety.

### TECHNICAL FIELD

The present invention pertains to the field of biotechnology, specifically relating to a strain that promotes seed germination of the genus Cremastra and applications thereof.

### BACKGROUND

Cremastra appendiculata (D. Don) Makino is a species within the genus Cremastra of the Orchidaceae family. It has very stringent requirements for its growth environment, typically thriving on the slopes or rock crevices of deep mountain valleys with excellent drainage and water retention. Due to the increasingly harsh ecological conditions, natural growth of Cremastra appendiculata in the wild has become extremely difficult. In recent years, rampant and uncontrolled harvesting has led to a sharp annual decline in wild populations. Currently, Cremastra appendiculata is classified as a Class II key protected wild plant in China and was listed in the “List of National Key Protected Wild Plants” (Second Batch) in 2021, necessitating urgent protection of its wild resources. Cremastra appendiculata possesses significant medicinal value, with its pseudobulb being the primary medicinal part. According to the “Mingyi Bie Lu” (Records of Famous Physicians), it has effects such as clearing heat and detoxifying, activating blood and removing stasis, moisturizing the lungs, and stopping coughs. Modern pharmacological studies have demonstrated that it exhibits strong anti-tumor, anti-inflammatory, and antioxidant activities, thereby garnering widespread attention. It holds substantial scientific research and economic value.

Due to the general absence of endosperm in Orchidaceae seeds, most rely on fungi to promote germination. Therefore, under natural conditions, the seeds of Cremastra appendiculata have great difficulty germinating on their own and have a long growth cycle. Currently, Cremastra appendiculata is primarily propagated through asexual reproduction of pseudobulbs and tissue culture of capsule seeds. These methods have low propagation rates, consistently limiting the large-scale cultivation of Cremastra appendiculata, making it difficult to meet species protection requirements and commercial demands, resulting in extremely high prices for medicinal materials.

In summary, developing a strain capable of promoting seed germination of the genus Cremastra plays a crucial role in increasing the germination rate of its seeds, thereby laying the foundation for the rational protection of its resources and large-scale cultivation.

### SUMMARY

The present invention primarily addresses the aforementioned technical problems by providing a strain that promotes seed germination of Cremastra genus plants and by offering specific application methods for this strain in promoting seed germination of Cremastra genus plants.

Specifically, the present invention provides the following technical solutions:

According to the first aspect, a symbiotic fungal strain for promoting seed germination of Orchidaceae plants is provided, where the ITS sequence of the strain comprises a sequence having 99% or higher identity with the sequence shown in SEQ ID NO:1.

In a preferred embodiment, the ITS sequence of the strain comprises the sequence shown in SEQ ID NO:1.

In a preferred embodiment, the symbiotic fungal strain for promoting seed germination of Orchidaceae plants belongs to the genus Coprinellus, and is deposited on Jun. 12, 2023, at the China Typical Culture Collection Center, Wuhan, Hubei Province, China. The deposit address is China Typical Culture Collection Center, Wuhan University, No. 8 Road, Wuchang District, Wuhan City, Hubei Province, and the strain deposit number is CCTCC_NO: M2023983.

Furthermore, the plant is a member of the Tribe Aerangideae.

Furthermore, the plant is a member of the genus Cremastra.

Furthermore, the plant is Cremastra appendiculata (D. Don) Makino.

According to the second aspect, the present application provides an application of any one of the aforementioned strains in promoting seed germination of Orchidaceae plants.

Furthermore, the plant is a member of the Tribe Aerangideae.

Furthermore, the plant is a member of the genus Cremastra.

Furthermore, the plant is Cremastra appendiculata (D. Don) Makino.

According to the third aspect, the present application provides an application of any one of the aforementioned strains in promoting the formation of protocorms from Orchidaceae plant seeds.

Furthermore, the plant is a member of the Tribe Aerangideae.

Furthermore, the plant is a member of the genus Cremastra.

Furthermore, the plant is Cremastra appendiculata (D. Don) Makino.

According to the fourth aspect, the present application provides a method for promoting germination of Orchidaceae plant seeds, which includes the following steps:
combining a culture containing any one of the strains mentioned above with a seed germination substrate to form a substrate containing the culture;
contacting the seeds with the substrate containing the culture; and
maintaining the substrate in a moist state.

In a preferred embodiment, the culture comprises only the strain.

Furthermore, the combination of the culture and the seed germination substrate is performed by mixing the culture into the substrate to form a substrate containing the culture.

Furthermore, the contacting of the seeds with the substrate containing the culture is performed by spreading the seeds into the substrate.

Furthermore, maintaining the substrate in a moist state is achieved by spraying water to keep the substrate moist.

Furthermore, the plant is a member of the Tribe Aerangideae.

Furthermore, the plant is a member of the genus Cremastra.

Furthermore, the plant is Cremastra appendiculata (D. Don) Makino.

According to the fifth aspect, the present application provides a method for promoting the formation of protocorms from Orchidaceae plant seeds, which the following steps:
combining a culture containing any one of the strains mentioned above with a seed germination substrate to form a substrate containing the culture;
contacting the seeds with the substrate containing the culture; and
maintaining the substrate in a moist state.

In a preferred embodiment, the culture includes only the strain.

Furthermore, the combination of the culture and the seed germination substrate is performed by mixing the culture into the substrate to form a substrate containing the culture.

Furthermore, the contacting of the seeds with the substrate containing the culture is performed by spreading the seeds into the substrate.

Furthermore, maintaining the substrate in a moist state is achieved by spraying water to keep the substrate moist.

Furthermore, the plant is a member of the Tribe Aerangideae.

Furthermore, the plant is a member of the genus Cremastra.

Furthermore, the plant is Cremastra appendiculata (D. Don) Makino.

According to the sixth aspect, the present application provides a DNA barcode for identifying fungi of the genus Coprinellus, wherein the DNA barcode includes the sequence as set forth in SEQ ID NO:1, or a sequence that is the reverse complement of the sequence set forth in SEQ ID NO:1.

Furthermore, the Coprinellus fungus is the strain deposited on Jun. 12, 2023, at the China Typical Culture Collection Center, Wuhan, Hubei Province, China, with the deposit address at China Typical Culture Collection Center, Wuhan University, No. 8 Road, Wuchang District, Wuhan City, Hubei Province, and the strain deposit number is CCTCC_NO: M2023983.

According to the sixth aspect, the present application also provides an application of the DNA barcode in identifying fungi of the genus Coprinellus.

According to the seventh aspect, the present application provides a method for identifying fungi of the genus Coprinellus, which includes:
extracting genomic DNA from the fungus to be tested;
performing PCR using the primers ITS1 5′-TCCGTAGGTGAACCTGCGG-3′ and ITS4 5′-TCCTCCGCTTATTGATATGC-3′;
sequencing the PCR products; and
determining the specific species of the fungus based on the sequencing results, wherein if the sequencing results are consistent with the DNA barcode sequence of claim 6, the tested fungus is determined to belong to the same genus as the strain deposited on Jun. 12, 2023, at the China Typical Culture Collection Center, Wuhan, Hubei Province, China, with the deposit address at China Typical Culture Collection Center, Wuhan University, No. 8 Road, Wuchang District, Wuhan City, Hubei Province, and the strain deposit number CCTCC_NO:M2023983.

Advantages and effects of the present application compared to the prior art are as follows:

First, the present invention provides a novel strain that can significantly promote the germination of seeds of the genus Cremastra.

Second, the present invention provides a novel strain that can improve the efficiency of converting seeds of the genus Cremastra into protocorms.

Third, the use of the present invention can promote the germination of seeds of the genus Cremastra and enhance the efficiency of converting seeds of the genus Cremastra into protocorms, thereby laying the foundation for the rational protection of its resources and large-scale cultivation.

### BRIEF DESCRIPTION OF THE DRAWINGS

FIG. 1 shows the colony morphology characteristics of the strain isolated by the present invention (deposit number CCTCC_NO:M2023983) cultured on PDA plates.

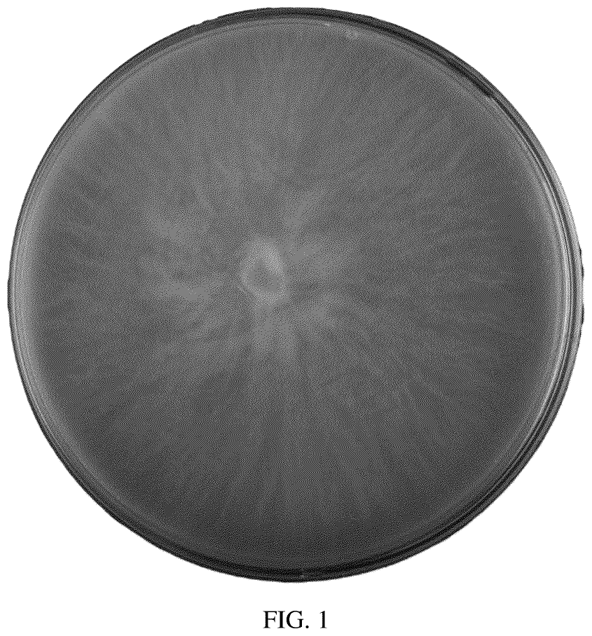

FIG. 2 shows photographs of protocorms obtained after inoculating Cremastra seeds with the strain of the present invention, specifically, FIG. 2A is a protocorm of seeds that germinated in the substrate after 2 months of inoculation, and FIGS. 2B to 2D are protocorms of sizes corresponding to 2 months, 3 months, and 5 months of inoculation, respectively.

FIG. 3 shows photographs of protocorms inoculated with other strains and blank controls after 2 months, showing no seed germination. Specifically, FIGS. 3A to 3G correspond to strains YSY-06, YSY-07, YSY-S-08, YSY-12, YSY-15, YSY-18, and blank control, respectively.

### DETAILED DESCRIPTION

To make the objectives, technical solutions, and beneficial technical effects of the present invention clearer, the following detailed description is provided in conjunction with specific embodiments. It should be understood that the following embodiments are provided solely for illustrative purposes and are not intended to limit the scope of the invention. Those skilled in the art may make various modifications and substitutions without departing from the spirit and scope of the invention. The experimental methods used in the following embodiments are conventional unless otherwise specified. The materials and reagents used in the following embodiments are commercially available unless otherwise specified.

### Embodiment 1: Isolation, Purification, and Identification of the Strain

Isolation and Purification of the Strain: Decayed soil beneath the tubers of wild Cremastra appendiculata (D. Don) Makino was collected from the Mazongling National Nature Reserve in Jinzhai County, Lu'an City, Anhui Province. Most impurities were manually selected and removed from the soil. The cleaned soil was then inoculated onto prepared Potato Dextrose Agar (PDA) medium within a laminar flow hood. Three plates were inoculated as primary cultures. The inoculated plates were placed in a 28° C. constant-temperature incubator for dark cultivation. Every three days, based on the differing morphology and size of new colonies, distinct colonies (including the corresponding regions of the solid medium) were aseptically cut using sterile scalpels and transferred to new PDA media for successive purification culturing. This process was repeated until pure colonies were obtained. The isolated strain was designated as YSYO1. The isolation medium used was PDA medium, purchased from Guangdong Huankai Microbial Technology Co., Ltd., and was prepared by adding 1 liter of deionized water to 40.1 grams of the medium according to the manufacturer's instructions.

### Strain Identification

1. Morphological Observation: As described above, a symbiotic fungal strain capable of promoting the germination of seeds of the genus Cremastra is provided, the biological characteristics of the fungus are as follows: On PDA (Potato Dextrose Agar) medium, the colony growth rate is 5.1±0.15 mm/day. The colonies exhibit well-developed aerial mycelia, with dense and relatively thick mycelia. The primary mycelia are white, and after approximately 10 days, the colonies display a color ranging from light yellow to deep yellow. The colony edges are neat, and the central portion is slightly thicker than the edges. The aging mycelia appear yellow-brown and sparse. After about 10 days of growth, a ring structure in the mycelia is observable.

2. Molecular Identification:

(1) Extraction of Fungal Genomic DNA: genomic DNA of the fungus was extracted using the SoLarbio Fungal Genomic DNA Extraction Kit. The concentration and purity of the extracted DNA were measured using a multifunctional microplate reader.

(2) Primer Design: the ITS (Internal Transcribed Spacer) sequences of ribosomal DNA were provided by Tongyong Bio (Anhui) Co., Ltd. The primer sequences used are: ITS1: 5′-TCCGTAGGTGAACCTGCGG-3′ and ITS4: 5′-TCCTCCGCTTATTGATATGC-3′.

(3) PCR Amplification System:

| Reagent | Volume (μL) |
|---|---|
| DNA | 2 |
| Enzyme (2 × Tq Plus Master Mix II) | 10 |
| Primer F | 0.5 |
| Primer R | 0.5 |
| Double-distilled water | 7 |

(4) PCR Cycling Conditions:

| | | |
|---|---|---|
| Temperature (° C.) | Time | Cycles |

| | | | |
|---|---|---|---|
| 95 | 5 | min | 35 cycles |
| 95 | 30 | sec | |
| 60 | 30 | sec | |
| 72 | 1 | min | |
| 72 | 10 | min | |

| | |
|---|---|
| 4 | ∞ |

(5) Sequencing Results: molecular sequencing and strain identification were conducted by Tongyong Bio (Anhui) Co., Ltd. The ITS sequences obtained from the sequencing results were compared for homology via BLAST on NCBI, with similarity results of 99% or higher used as the standard for molecular identification of the strain. The identification result was determined to be Coprinellus (genus Coprinellus).

The strain isolated in this embodiment was deposited on Jun. 12, 2023, at the China Typical Culture Collection Center, Wuhan, Hubei Province, China. The deposit address is China Typical Culture Collection Center, Wuhan University, No. 8 Road, Wuchang District, Wuhan City, Hubei Province, and the strain deposit number is CCTCC_NO:M2023983.

(SEQ ID NO. 1)
5'-GCGGAAGGATCATTAACGAATAACTATGGTGTCTTGGTTGTA
GCTGGCTCCTCGGAGCATTGTGCACGCCCGCCATTTTTATCTAT
CCACCTGTGCACCGACTGTAGGTCTGGATGACTCTCGTGCTCTCT
GAGTGCGGATGCGAGGATTGCCCTTCAACTCGGAGGTGTCTCTCC
TCGAATTTCCAGGCTCTACGTCTTTTTACAAACCCCAAAAGCATG
ATATAGAATGTAGTCAATGGGCTTGATCGCCTATAAAACACTATA
CAACTTTCAGCAACGGATCTCTTGGCTCTCGCATCGATGAAGAAC
GCAGCGAAATGCGATAAGTAATGTGAATTGCAGAATTCAGTGAAT
CATCGAATCTTTGAACGCACCTTGCGCTCCTTGGTATTCCGAGGA
GCATGCCTGTTTGAGTGTCATTAAATTCTCAACCTCACCCGTTTT
CTGAACGGTTCTCCGAGGCTTGGATGTGGGGGTTTGTGCAGGCTG
CCTCAGCGCGGTCTGCTCCCCTGAAATGCATTAGCGAGTTCGTAC
TGAGCTCCGTCTATTGGTGTGATAATTATCTACGCCGTGGATAGG
GTTTAGACTCGCTTCTAACCGTCCGCAAGGACAATACCTTTGACA
ATTTGACCTCAAATCAGGTAGGACTACCCGCTGAACTTAAGCAT
A-3'.

### Embodiment 2: Comparison of Coprinellus Fungal Strains in Promoting Germination of Cremastra appendiculata (D. Don) Makino Seeds in Petri Dishes

To verify that the Coprinellus fungal strain isolated by the present invention has the ability to promote germination of Cremastra appendiculata (D. Don) Makino seeds, six additional Coprinellus fungal strains were selected to conduct a comparative test with the YSYO1 strain obtained by the present invention. The seven strains are YSY01 (obtained by the present invention), YSY-06, YSY-07, YSY-S-08, YSY-12, YSY-15, and YSY-18. The latter six strains are derived from strains previously isolated and identified by this laboratory, and the ITS sequences identified in the preliminary sequencing identification are as follows:

YSY-06:
CTTCTCTTGCTTTAGTAGGGGCCTGCGGAAGGATCATTAACGAA
TAACTATGGTGTCTTGGTTGTAGCTGGCTCCTCGGA
GCATTGTGCACGCCCGCCATTTTTATCTATCCACCTGTGCACCG
ACTGTAGGTCTGGATGACTCTCGTGCTCTCTGAGTG
CGGATGCGAGGATTGCCCTTCAACTCGGAGGTGTCTCTCCTCGA
ATTTCCAGGCTCTACGTCTTTTTACACACCCCAAAA
GCATGATATAGAATGTAGTCAATGGGCTTGATCGCCTATAAAA
CACTATACAACTTTCAGCAACGGATCTCTTGGCTCTC
GCATCGATGAAGAACGCAGCGAAATGCGATAAGTAATGTGAAT
TGCAGAATTCAGTGAATCATCGAATCTTTGAACGCAC
CTTGCGCTCCTTGGTATTCCGAGGAGCATGCCTGTTTGAGTGTC
ATTAAATTCTCAACCTCACCCGTTTTCTGAACGGTT
CTCCGAGGCTTGGATGTGGGGGTTTGTGCAGGCTGCCTCAGCGC
GGTCCGCTCCCCTGAAATGCATTAGCGAGTTCGTAC
TGAGCTCCGTCTATTGGTGTGATAATTATCTACGCCGTGGACAG
GGTTTAGACTCGCTTCTAACCGTCCGCAAGGACAAT
ACCTTTGACAATTTGACCTCAAATCAGGTAGGACTACCCGCTGA
ACTTAAGCATATCAAAAAGGGAGAGAAAA
YSY-07:
GGTCCGTAGGAGAACCTGCGGAAGGATCATTAACGAATAACTA
TGGTGTCTTGGTTGTCGCTGGCTCCTCGGAGCATCGT
GCACGCCCGCCATTTTTATCTATCCACCTGTGCACCGAATGTAG
GTCTGGATGACTCTCGCCTCCGGGCGGATGCGAGGT
TTGCGTGCGAGCGCTCTCCTCGAATTTCCAGGCTCTACGTCTCTT
TACACACCCCAAACGTATGATGCAGAATGTAGTCA
ATGGGCCTTTACAAGCCTATAAAACACTATACAACTTTCAGCAA
CGGATCTCTTGGCTCTCGCATCGATGAAGAACGCAG
CGAAATGCGATAAGTAATGTGAATTGCAGAATTCAGTGAATCA
TCGAATCTTTGAACGCACCTTGCGCTCCTTGGTATTC
CGAGGAGCATGCCTGTTTGAGTGTCATTAAATTCTCAACCTCAC
CGGTTTTCTGAACCGTTCTCCGAGGCTTGGATGTGG
GGGTCTGTGCAGGTCGCCTCAGCGCGGTCTGCTCCCCTGAAATG
CATTAGCGAGATTCATTCTGGACCTCCGTCTATCGG
TGTGATAATTATCTACGTCGTTGACTTGGTTCGGACTCGCTTCTA
ACCGTCCGCGAGGACAACATACTTGACAATTTGAC
TCAAATCAGGTAGGACTACCCGCTGAACTTAAGCATATCAATA
AGCGGAGGGAAA
YSY-08:
TTTTTTCGGGGGGGGCTGCGGTAGGATCATTAACGAATAACTAT
GGCTTTGGTTGTAGCTGGCTCCTCGGAGCAATGTGC
ACGCCCGCCATTTTTATCTTTCCACCTGTGCACCGACTGTAGGT
CTGGATACCTCTCGCCTCCGGGCGGATGCGAGGGTT
GCTCGTAAGGGCTTCCCTTGAACTTCCAGGCTCTACGTCTTTTTA
CACACCCCAATAGTATGATGCAGAATGTAGTCAAT
GGGCTTCTCAGCCTATAAAACACTATACAACTTTCAGCAACGGA
TCTCTTGGCTCTCGCATCGATGAAGAACGCAGCGAA
ATGCGATAAGTAATGTGAATTGCAGAATTCAGTGAATCATCGA
ATCTTTGAACGCACCTTGCGCTCCTTGGTATTCCGAG
GAGCATGCCTGTTTGAGTGTCATTAAATTCTCAACCTCACCAGT
TTTCTGAACTGACTGAGGCTTGGATGTGGGGGTTTG
TGCAGGCTGCCTCACCGGCGGTCTGCTCCCCTGAAATGCATTAG
CGAGGTTCATGCTGGACCTCCGTCTATTGGTGTGAT
AATTATCTACGCCGTGGACGAGGATACAGACTCGCTTCTAACCG
TCCGCGAGGACAATACCTTGACAATTTGACCTCAAA
TCAGGTAGGACTACCCGCTGAACTTAAGCATTTTAAGGGGGGG
GGAAGAAAAA
YSY-12:
CTTTTTTTCGGGGGGGGGTCGGAAGGATCATTTACGAATAACTA
TGGCGTTGGTTGTAGCTGGCTCCTCGGAGCAATGTG
CACGCCCGCCATTTTTATCTTTCCACCTGTGCACCGACTGTAGG
TCTGGATACCTCTCGCCTCCGGGCGGATGCGAGGGT
TGCTCGTAAGGGCTTCCCTTGAACTTCCAGGCTCTACGTCTTTTT
ACACACCCCAATAGTATGATGCAGAATGTAGTCAA
TGGGCTTCTCAGCCTATAAAACACTATACAACTTTCAGCAACGG
ATCTCTTGGCTCTCGCATCGATGAAGAACGCAGCGA
AATGCGATAAGTAATGTGAATTGCAGAATTCAGTGAATCATCG
AATCTTTGAACGCACCTTGCGCTCCTTGGTATTCCGA
GGAGCATGCCTGTTTGAGTGTCATTAAATTCTCAACCTCACCAG
TTTTCTGAACTGACTGAGGCTTGGATGTGGGGGTTT
GTGCAGGCTGCCTCACCGGCGGTCTGCTCCCCTGAAATGCATTA
GCGAGGTTCATGCTGGACCTCCGTCTATTGGTGTGA
TAATTATCTACGCCGTGGACGAGGATACAGACTCGCTTCTAACC
GTCCGCGAGGACAATACCTTGACAATTTGACCTCAA
ATCAGGTAGGACTACCCCCTGAACTTAAGCATTTAAAAGGGGG
GGGGGGGAA
YSY-15:
CTTTCGGTAGGGGGAACCTGCGGAAGGATCATTAACGAATAAC
TATGGCGTTGGTTGTAGCTGGCTCCTCGGAGCAATGT
GCACGCCCGCCATTTTTATCTTTCCACCTGTGCACCGACTGTAG
GTCTGGATACCTCTCGCCTCCGGGCGGATGCGAGGG
TTGCTCGTAAGGGCTTCCCTTGAACTTCCAGGCTCTACGTCTTTT
TACACACCCCAATAGTATGATGCAGAATGTAGTCA
ATGGGCTTCTCAGCCTATAAAACACTATACAACTTTCAGCAACG
GATCTCTTGGCTCTCGCATCGATGAAGAACGCAGCG
AAATGCGATAAGTAATGTGAATTGCAGAATTCAGTGAATCATC
GAATCTTTGAACGCACCTTGCGCTCCTTGGTATTCCG
AGGAGCATGCCTGTTTGAGTGTCATTAAATTCTCAACCTCACCA
GTTTTCTGAACTGACTGAGGCTTGGATGTGGGGGTT
TGTGCAGGCTGCCTCACCGGCGGTCTGCTCCCCTGAAATGCATT
AGCGAGGTTCATGCTGGACCTCCGTCTATTGGTGTG
ATAATTATCTACGCCGTGGACGAGGATACAGACTCGCTTCTAAC
CGTCCGCGAGGACAATACCTTGACAATTTGACCTCA
AATCAGGTAGGACTACCCGCTGAACTTAAGCATATCAATAGGC
CCGGAGGAA
YSY-18:
CTTCCGTTGGGGGGGCCTGCGGAAGGATCATTAACGAATAACT
ATGGCGTTGGTTGTAGCTGGCTCCTCGGAGCAATGTG
CACGCCCGCCATTTTTATCTTTCCACCTGTGCACCGACTGTAGG
TCTGGATACCTCTCGCCTCCGGGCGGATGCGAGGGT
TGCTCGTAAGGGCTTCCCTTGAACTTCCAGGCTCTACGTCTTTTT
ACACACCCCAATAGTATGATGCAGAATGTAGTCAA
TGGGCTTCTCAGCCTATAAAACACTATACAACTTTCAGCAACGG
ATCTCTTGGCTCTCGCATCGATGAAGAACGCAGCGA
AATGCGATAAGTAATGTGAATTGCAGAATTCAGTGAATCATCG
AATCTTTGAACGCACCTTGCGCTCCTTGGTATTCCGA
GGAGCATGCCTGTTTGAGTGTCATTAAATTCTCAACCTCACCAG
TTTTCTGAACTGACTGAGGCTTGGATGTGGGGGTTT
GTGCAGGCTGCCTCACCGGCGGTCTGCTCCCCTGAAATGCATTA
GCGAGGTTCATGCTGGACCTCCGTCTATTGGTGTGA
TAATTATCTACGCCGTGGACGAGGATACAGACTCGCTTCTAACC
GTCCGCGAGGACAATACCTTGACAATTTGACCTCAA
ATCAGGTAGGACTACCCGCTGAACTTAAGCATATCAAAGGGGC
GAAAGGAA

### 2.1 Materials

Oat Meal Agar (OMA), Cremastra appendiculata (D. Don) Makino fruits, and culture bags containing seven different fungal strains.

### 2.2 Methods

2.2.1 Preparation of Symbiotic Germination Medium: the medium used was Oat Meal Agar (OMA, 4 g/L oatmeal powder, 8 g/L agar, pH natural). After preparing the medium, it was sterilized at 121° C. for 20 minutes and set aside for use.

2.2.2 Sterilization and Disinfection of Cremastra appendiculata (D. Don) Makino Seeds:

The surface of mature Cremastra. appendiculata (D. Don) Makino capsules was disinfected with 75% ethanol for 30 seconds and rinsed three times with sterile water. The capsule shells were then opened to release the seeds, which were wrapped with sterile filter paper, disinfected again with 75% ethanol for 30 seconds, and rinsed three times with sterile water. Subsequently, the seeds were disinfected with 1% sodium hypochlorite for 3 minutes and rinsed five times with sterile water before use.

2.2.3 Sowing and Culturing: the OMA medium cooled to 40-50° C. was poured into sterile Petri dishes (9 cm in diameter), approximately 20 mL per dish. After solidification, sterile filter paper with a central hole was placed on the surface. Cremastra appendiculata (D. Don) Makino seeds were uniformly dispersed in sterile water. Using a stereomicroscope, the seed concentration in the sterile water was determined and diluted continuously to achieve a seed concentration of approximately 15 seeds/0.1 mL. Using a sterile swab, 1 mL of the uniformly dispersed seed-containing sterile water was evenly spread onto the filter paper. Subsequently, mycelia from the seven purified fungal strains were inoculated into the central hole of the filter paper. The Petri dishes were sealed with sealing film. A blank control group without fungal inoculation was also prepared. Each treatment was replicated five times and cultured in an artificial climate chamber at 22° C.

### 2.2.4 Detection:

After sowing, the germination of seeds was observed weekly under a stereomicroscope. The number of germinated seeds at different germination stages was recorded and statistically analyzed (seed germination was marked by the swelling of the embryo breaking through the seed coat). The statistical results are shown in Table 1.

TABLE 1
Germination Rate of Cremastra appendiculata (D.
Don) Makino Seeds under Different Treatments

| | Germination Rate of Cremastra |
|---|---|
| Treatment Method | appendiculata (D. Don) Makino Seeds |
| No symbiotic | 0 |
| fungal strain | |
| YSY01 | 85% |
| YSY-06 | 0 |
| YSY-07 | 0 |
| YSY-08 | 0 |
| YSY-12 | 0 |
| YSY-15 | 0 |
| YSY-18 | 0 |

### Embodiment 3: Comparison of Coprinellus Fungal Strains in Promoting Germination of Cremastra appendiculata (D. Don) Makino Seeds in Decayed Mixed Hardwood Leaf Substrate

To further explore whether the strains of the present invention can promote the germination of Cremastra appendiculata (D. Don) Makino seeds under natural environmental conditions, we conducted symbiotic germination experiments using decayed mixed hardwood leaves as the substrate. These experiments were carried out in January 2023 in Lu'an City, Anhui Province, under outdoor conditions with natural light cycles and temperature variations.

### 3.1 Materials

Decayed mixed hardwood leaves, Cremastra appendiculata (D. Don) Makino fruits, and culture bags containing seven different fungal strains.

### 3.2 Methods

3.2.1 collected decayed mixed hardwood leaves were placed in a sterilizer at 120° C. for 30 minutes and allowed to cool for later use.

3.2.2 different fungal strain culture bags were taken, and the fungal material was mixed into the decayed mixed hardwood leaves separately. This mixture served as the substrate material for seed germination cultivation.

3.2.3 opened the intact Cremastra appendiculata (D. Don) Makino fruits and evenly scattered the seeds into the substrate. Seeds from each fruit were uniformly spread over 1 m² of substrate, topped with a thin layer of mixed hardwood leaves. Regularly sprayed with clean water to maintain the substrate in a moist state.

3.2.4 the substrate containing both the fungal strains and Cremastra appendiculata (D. Don) Makino seeds was placed outdoors under natural light cycles. The germination status of the seeds was regularly observed.

Due to the extremely small size of Cremastra appendiculata (D. Don) Makino seeds, accurate germination rates could not be determined by counting seeds directly under a microscope. Instead, the number of protocorms germinated within a single fruit was observed and counted as the basis for evaluating germination rates.

### 3.3 Results

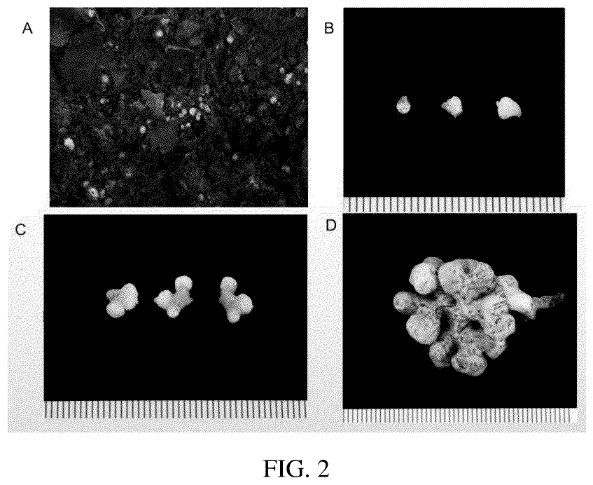

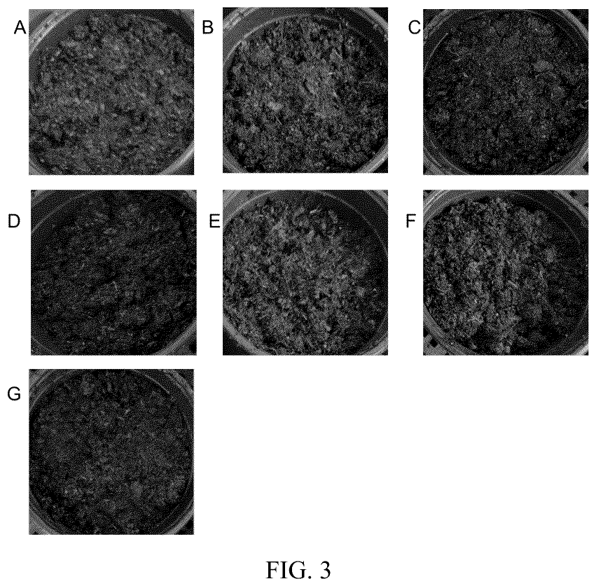

The results showed that the fungal strain numbered YSYO1 obtained by the present invention significantly promoted the germination of Cremastra appendiculata (D. Don) Makino seeds. After 4 weeks of sowing, protocorms visibly enlarged to 1-2 mm in size with noticeable mycorrhizae on their surfaces. After 8 weeks, the protocorms expanded to 4-5 mm, developing branches that formed irregular polygonal shapes. After 20 weeks of sowing, the protocorms reached diameters of 2-3 cm, producing multiple branches with irregular shapes (FIG. 2). In the substrate mixed with the YSYO1 strain, each Cremastra appendiculata (D. Don) Makino fruit produced 1,500-2,000 protocorms (Table 2). No germination of Cremastra appendiculata (D. Don) Makino seeds was observed with other fungal strains (see FIG. 3 and Table 2).

TABLE 2
Statistical Results of Protocorms Produced per Cremastra appendiculata
(D. Don) Makino Fruit under Different Treatments

| | Number of Protocorms per |
|---|---|
| Treatment Method | Cremastra appendiculata Fruit |
| No symbiotic | 0 |
| fungal strain | |
| YSY01 | 1500-2000 |
| YSY-06 | 0 |
| YSY-07 | 0 |
| YSY-08 | 0 |
| YSY-12 | 0 |
| YSY-15 | 0 |
| YSY-18 | 0 |

In summary, the strains of the present invention can significantly enhance the germination rate of Cremastra appendiculata (D. Don) Makino seeds. This advancement lays a foundation for the rational conservation of Cremastra appendiculata resources and the large-scale cultivation of this species.

Based on the disclosures and teachings of the above description, those skilled in the art can make appropriate changes and modifications to the above embodiments. Therefore, the present invention is not limited to the specific embodiments disclosed and described above, and certain modifications and changes to the present invention should also fall within the scope of the claims. Furthermore, although some specific terms are used in this description for convenience, these terms are not intended to limit the present invention in any way.

What is claimed is:

1. A symbiotic fungal strain for promoting germination of Cremastra genus plant seeds, characterized in that the symbiotic strain belongs to the genus Coprinellus, deposited on Jun. 12, 2023, at the China Typical Culture Collection Center, Wuhan, Hubei Province, China, with the deposit address at China Typical Culture Collection Center, Wuhan University, No. 8 Road, Wuchang District, Wuhan City, Hubei Province, and the strain deposit number is CCTCC_NO: M2023983.

2. A method for promoting germination of Cremastra genus plant seeds, characterized by comprising the following steps:
combining a culture containing the strain of claim 1 with a seed germination substrate to form a substrate containing the culture;
contacting the seeds with the substrate containing the culture; and
maintaining the substrate in a moist state.

3. A DNA barcode for identifying the symbiotic strain of claim 1, wherein the DNA barcode is the sequence as set forth in SEQ ID NO:1, or a sequence that is the reverse complement of the sequence set forth in SEQ ID NO:1.


SEQUENCE LISTING (7 sequences)

SEQ ID NO: 1 - DNA, 671 bp, Coprinellus sp.
  source 1..671: mol_type = genomic DNA
gcggaaggatcattaacgaataactatggtgtcttggttgtagctggctcctcggagcattgtgcacgcccgccatttttatctatccacctgtgcaccgactgtaggtctggatgactctcgtgctctctgagtgcggatgcgaggattgcccttcaactcggaggtgtctctcctcgaatttccaggctctacgtctttttacaaaccccaaaagcatgatatagaatgtagtcaatgggcttgatcgcctataaaacactatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaacctcacccgttttctgaacggttctccgaggcttggatgtgggggtttgtgcaggctgcctcagcgcggtctgctcccctgaaatgcattagcgagttcgtactgagctccgtctattggtgtgataattatctacgccgtggatagggtttagactcgcttctaaccgtccgcaaggacaatacctttgacaatttgacctcaaatcaggtaggactacccgctgaacttaagcata

SEQ ID NO: 2 - DNA, 713 bp, Coprinellus sp.
  source 1..713: mol_type = genomic DNA
cttctcttgctttagtaggggcctgcggaaggatcattaacgaataactatggtgtcttggttgtagctggctcctcggagcattgtgcacgcccgccatttttatctatccacctgtgcaccgactgtaggtctggatgactctcgtgctctctgagtgcggatgcgaggattgcccttcaactcggaggtgtctctcctcgaatttccaggctctacgtctttttacacaccccaaaagcatgatatagaatgtagtcaatgggcttgatcgcctataaaacactatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaacctcacccgttttctgaacggttctccgaggcttggatgtgggggtttgtgcaggctgcctcagcgcggtccgctcccctgaaatgcattagcgagttcgtactgagctccgtctattggtgtgataattatctacgccgtggacagggtttagactcgcttctaaccgtccgcaaggacaatacctttgacaatttgacctcaaatcaggtaggactacccgctgaacttaagcatatcaaaaagggagagaaaa

SEQ ID NO: 3 - DNA, 695 bp, Coprinellus sp.
  source 1..695: mol_type = genomic DNA
ggtccgtaggagaacctgcggaaggatcattaacgaataactatggtgtcttggttgtcgctggctcctcggagcatcgtgcacgcccgccatttttatctatccacctgtgcaccgaatgtaggtctggatgactctcgcctccgggcggatgcgaggtttgcgtgcgagcgctctcctcgaatttccaggctctacgtctctttacacaccccaaacgtatgatgcagaatgtagtcaatgggcctttacaagcctataaaacactatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaacctcaccggttttctgaaccgttctccgaggcttggatgtgggggtctgtgcaggtcgcctcagcgcggtctgctcccctgaaatgcattagcgagattcattctggacctccgtctatcggtgtgataattatctacgtcgttgacttggttcggactcgcttctaaccgtccgcgaggacaacatacttgacaatttgactcaaatcaggtaggactacccgctgaacttaagcatatcaataagcggagggaaa

SEQ ID NO: 4 - DNA, 693 bp, Coprinellus sp.
  source 1..693: mol_type = genomic DNA
ttttttcggggggggctgcggtaggatcattaacgaataactatggctttggttgtagctggctcctcggagcaatgtgcacgcccgccatttttatctttccacctgtgcaccgactgtaggtctggatacctctcgcctccgggcggatgcgagggttgctcgtaagggcttcccttgaacttccaggctctacgtctttttacacaccccaatagtatgatgcagaatgtagtcaatgggcttctcagcctataaaacactatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaacctcaccagttttctgaactgactgaggcttggatgtgggggtttgtgcaggctgcctcaccggcggtctgctcccctgaaatgcattagcgaggttcatgctggacctccgtctattggtgtgataattatctacgccgtggacgaggatacagactcgcttctaaccgtccgcgaggacaataccttgacaatttgacctcaaatcaggtaggactacccgctgaacttaagcattttaagggggggggaagaaaaa

SEQ ID NO: 5 - DNA, 692 bp, Coprinellus sp.
  source 1..692: mol_type = genomic DNA
ctttttttcgggggggggtcggaaggatcatttacgaataactatggcgttggttgtagctggctcctcggagcaatgtgcacgcccgccatttttatctttccacctgtgcaccgactgtaggtctggatacctctcgcctccgggcggatgcgagggttgctcgtaagggcttcccttgaacttccaggctctacgtctttttacacaccccaatagtatgatgcagaatgtagtcaatgggcttctcagcctataaaacactatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaacctcaccagttttctgaactgactgaggcttggatgtgggggtttgtgcaggctgcctcaccggcggtctgctcccctgaaatgcattagcgaggttcatgctggacctccgtctattggtgtgataattatctacgccgtggacgaggatacagactcgcttctaaccgtccgcgaggacaataccttgacaatttgacctcaaatcaggtaggactaccccctgaacttaagcatttaaaaggggggggggggaa

SEQ ID NO: 6 - DNA, 692 bp, Coprinellus sp.
  source 1..692: mol_type = genomic DNA
ctttcggtagggggaacctgcggaaggatcattaacgaataactatggcgttggttgtagctggctcctcggagcaatgtgcacgcccgccatttttatctttccacctgtgcaccgactgtaggtctggatacctctcgcctccgggcggatgcgagggttgctcgtaagggcttcccttgaacttccaggctctacgtctttttacacaccccaatagtatgatgcagaatgtagtcaatgggcttctcagcctataaaacactatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaacctcaccagttttctgaactgactgaggcttggatgtgggggtttgtgcaggctgcctcaccggcggtctgctcccctgaaatgcattagcgaggttcatgctggacctccgtctattggtgtgataattatctacgccgtggacgaggatacagactcgcttctaaccgtccgcgaggacaataccttgacaatttgacctcaaatcaggtaggactacccgctgaacttaagcatatcaataggcccggaggaa

SEQ ID NO: 7 - DNA, 691 bp, Coprinellus sp.
  source 1..691: mol_type = genomic DNA
cttccgttgggggggcctgcggaaggatcattaacgaataactatggcgttggttgtagctggctcctcggagcaatgtgcacgcccgccatttttatctttccacctgtgcaccgactgtaggtctggatacctctcgcctccgggcggatgcgagggttgctcgtaagggcttcccttgaacttccaggctctacgtctttttacacaccccaatagtatgatgcagaatgtagtcaatgggcttctcagcctataaaacactatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaacctcaccagttttctgaactgactgaggcttggatgtgggggtttgtgcaggctgcctcaccggcggtctgctcccctgaaatgcattagcgaggttcatgctggacctccgtctattggtgtgataattatctacgccgtggacgaggatacagactcgcttctaaccgtccgcgaggacaataccttgacaatttgacctcaaatcaggtaggactacccgctgaacttaagcatatcaaaggggcgaaaggaa